(19) United States
(12) Patent Application Publication
(10) Pub. No.: US 2018/0114196 A1
(43) Pub. Date: Apr. 26, 2018

(54) Systems and Methods for Managing a Patient of Medical Practice

(71) Applicant: AUTOMED KIOSK PTY LTD, Burwood, Victoria (AU)

(72) Inventor: Pietro Rocco Demaio, Burwood, Victoria (AU)

(21) Appl. No.: 15/568,667

(22) PCT Filed: Apr. 24, 2016
(86) PCT No.: PCT/AU2016/050295

§ 371 Date: Oct. 23, 2017

Related U.S. Application Data
(60) Provisional application No. 62/151,554, filed on Apr. 23, 2015.

(51) Int. Cl. G06Q 10/10; G16H 10/60

(52) U.S. Cl. CPC G06Q 10/1095; G06Q 50/10; G16H 10/60

(57) ABSTRACT

The present invention provides a system for managing a patient of a medical practice, the system comprising: patient identification means, a database comprising patient-specific information, a processor-based device in operable connection with the patient identification means and the patient database, and patient-viewable or patient-audible output means. Configurations of the system providing for accepting payments from a patient, allowing selection of a certain practitioner, and the making of follow-up appointments are also provided.

### FIELD OF THE INVENTION

The present invention is directed to computer-based systems for the management of a business. In particular, the invention is directed to the management of a medical practice.

### BACKGROUND TO THE INVENTION

Medical practices (and particularly practices having a high patient turnover) direct significant resources to handling patient arrivals, triaging patients, ensuring patient details are up to date, setting appointments, and improving productivity of the practitioner(s) on site. Further resources are directed to informing and encouraging patients to participating in current health programs.

Electronic customer handling kiosks are known in the art. Such kiosks typically issue the customer with a number of options on a video display unit to identify the service required, and directs the customer to a particular area capable of assisting with the need. Some kiosks also issue the customer with a number, to facilitate customers being attended to in order of arrival. More sophisticated kiosks present the customer with a site map with directions to a relevant area of the building.

While useful in many settings, existing customer handling kiosks do not provide any significant advantage to a medical practice. Most medical consultations are appointment-based and so patient order of arrival is generally irrelevant. While potentially useful, the use of maps would only improve the probability that patient is in the correct area when called by the medical practitioner into the consulting room. Many medical practices are not sufficiently large so as to require this feature.

Importantly, a prior art arrival kiosk would not significantly reduce the resources required to operate a medical practice nor to increase the efficiency of the practice. Furthermore, prior art kiosks would not improve the patient experience while visiting the practice.

It is an aspect of the present invention to overcome or ameliorate a problem of the prior art by providing systems and methods for managing patients of a medical practice. In another aspect, the present invention provides an alternative to prior art systems and methods.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In one aspect, but not necessarily the broadest aspect the present invention provides a system for managing a patient of a medical practice, the system comprising:
patient identification means,
a database comprising patient-specific information,
a processor-based device in operable connection with the patient identification means and the patient database, and
patient-viewable or patient-audible output means.

In one embodiment of the system, the patient identification means is configured to allow identification of the patient without the need for patient input.

In one embodiment of the system, the patient identification means is not reliant on patient input to identify the patient.

In one embodiment of the system, the patient identification means is capable of reading information from medical practice-issued identification means or third party-issued identification means.

In one embodiment of the system, the third party is a government or a health insurance company.

In one embodiment of the system, the patient identification means is capable of reading biometric information of the patient.

In one embodiment of the system, the patient identification means is capable of reading information from a personal electronic device of the patient.

In one embodiment of the system, the information obtained from the patient identification means is transmitted to the processor-based based device, the processor based-device interrogating the patient database to retrieve patient-specific information.

In one embodiment of the system, the patient-viewable display means displays general and/or patient-specific information.

In one embodiment of the system, the patient-specific information and/or general information relates to an appointment time, an appointment location, a practitioner identity, a practitioner characteristic, a patient preference, a fee, a suggested product or service, or a health alert.

In one embodiment of the system, the patient-specific information relates to a patient name, a patient contact detail, or a patient health insurance detail.

In one embodiment the system comprises patient-usable data input means, wherein the patient-viewable display means is configured to display a question and the patient-usable data input means is configured to receive a response by the patient.

In one embodiment of the system, the question is selected from a group of existing questions based on the patient identity and/or patient-specific information of the database.

In one embodiment, the system is configured to alert a second system of the medical practice, or medical practice personnel, that the patient has arrived.

In one embodiment of the system, the patient-usable input means and patient-viewable output means are configured to allow the patient to set a follow up appointment during or after a consultation.

In one embodiment of the system, the system further comprises means for reading information from a document related to an ancillary medical or paramedical service.

In one embodiment of the system, the patient identification means is a configured as a kiosk located in a reception or entry of a medical practice, the kiosk comprising visual display means.

In one embodiment, the system comprises payment means.

In one embodiment of the system, the payment means in on or proximal to the kiosk.

In one embodiment, the system is configured so as to receive a prepayment via the payment means.

In one embodiment, the system is configured so as to (i) store the prepayment electronically, (ii) allow a practitioner to enter a consultation charge, and (iii) calculating a total charge based on the amount of the stored prepayment and the consultation charge.

In one embodiment, the system is configured so as to allow a practitioner to reverse, rebate or otherwise return the prepayment to the patient.

In one embodiment the system comprises (i) printing means configured to issue an appointment document to the patient, and/or (ii) data transmission means configured to electronically transmit appointment information to a patient computer or a patient personal electronic device.

In a second aspect, the present invention provides a method for managing a patient of a medical practice, the method comprising the steps of:
providing the system as described herein, and
instructing or allowing a patient to utilise the patient identification means.

In a third aspect, the present invention provides media comprising processor-executable instructions configured to operate the system as described herein, or the method as described herein.

In a fourth aspect, the present invention comprises an electronic kiosk comprising patient identification means, the kiosk operable within the system as described herein, or the method as described herein.

In a fifth aspect, the present invention provides a document issued by a practitioner in the course of a medical consultation, the document comprising computer-readable means configured to facilitate the making of a follow-up appointment by way of a computer-implemented appointment system.

In one embodiment, the document comprises computer-readable means configured to identify the patient in the making of a follow-up appointment by way of a computer-implemented appointment system.

### BRIEF DESCRIPTION OF THE DRAWINGS

FIGS. 1 to 5 show diagrammatically preferred screen shots of a touch screen device of kiosks of the present invention.

FIG. 6 shows a block diagram of a system of the present invention.

### DETAILED DESCRIPTION OF THE INVENTION

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments covered by the claims.

Throughout the description and the claims of this specification the word “comprise” and variations of the word, such as “comprising” and “comprises” is not intended to exclude other additives, components, integers or steps.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

Applicant proposes that one or more problems of the prior art may be overcome or at least alleviated by providing means for a patient of a medical practice to be properly identified as a first step in managing the patient. Accordingly, in a first aspect, the present invention provides a system for managing a patient of a medical practice, the system comprising: patient identification means, a database comprising patient-specific information, a processor-based device in operable connection with the patient identification means and the patient database, and patient-viewable or patient-audible output means.

Applicant has discovered that advantage is provided in operating a medical practice where patient identification is assured (or at least improved) as a first step in managing the patient upon arrival. In prior art methods, upon arrival the patient approaches a reception area and is attended to by a staff member. In the course of that interaction, the patient identifies himself or herself verbally. In a significant number of interactions the identification process is protracted often due to language difficulties, the patient being known by more than one name, the patient having changed name, the existence of multiple spellings for a name etc. Even where due care is taken by the staff member, patient misidentification is not uncommon.

The present system and methods provides for the use of electronic patient identification means as a first step in managing the patient upon arrival at the practice. Typically, the patient identification means is a component of an arrival kiosk disposed about the entrance point of the practice. The kiosk is generally processor-controlled (for example by an incorporated person computer), and comprises output means including a video display screen viewable by the patient, or a speaker audible by the patient. In some embodiments the video display screen is touch sensitive allowing the patient to input data as required.

Upon arrival, the patient sights or hears the output means imparting an instruction (in text and/or graphical and/or audio form) to utilize the patient identification means before proceeding further.

While the patient identification means may rely on patient input (such as by text input, or voice recognition) it is preferred that the means does not rely on patient input. For example, the patient identification means may rely on an identification document issued by the practice itself, or by a third party.

In one embodiment, the system utilizes a medical practice-issued document (such as a card, an appointment card, or an adhesive-backed label that may be affixed to another document or a card, an invoice, a prescription, a referral letter or a pathology service request form) . The medical practice document may be configured to uniquely identify each patient of the practice. Graphical means such as a bar code or a QR code (implementation of both are known to the skilled artisan) may be applied in this regard. Laser-based readers of such codes are well known and may be incorporated into the system.

Alternatively, optical character recognition means may be used in the system to ascertain the patient's identification from a practice-issued document. The optical character recognition means may be configured to establish the identity of the patient by recognising the patient's name (in text) or a patient-specific number (in text) issued by the medical practice.

Other methods of encoding data capable of identifying a patient are also including within the scope of the invention including the use magnetic-based media (such as encoded strips or magnetic inks). Readers of such media are also known in the art, and therefore readily incorporated into the present systems. Where the system comprises a kiosk, the kiosk may therefore comprise an optical or magnetic based reader.

In some embodiments, the practice-issued patient identification means is not a document, and may be a purely electronic means. It is contemplated that a personal electronic device of the patient (such as a smart phone) may comprise practice-issued patient identification means. The screen of such devices can display a unique bar code or QR code or even simply a string of text which is optically recognised.

Alternatively, the patient's personal electronic device may transmit an electronic signal (such as by radio means including WiFi™, Bluetooth™or ANT™) to the patient identification means. In this embodiment, the data encoded in the signal is a unique patient identifier issued by the practice. A system configured in this way may allow for a patient to simply enter a practice, and without any interaction with an arrival kiosk to be identified by the system and recognized as having arrived.

In other embodiments, the system is configured to identify a patient based on means issued by a third party. Preferably, the third party issued means is a document that would normally be carried by a patient when entering a medical practice. As one example, a government issued health care card is used. Examples of such cards include the Medicare Card (Australia), the European Health Insurance Card (European Economic Area countries), and the Social Security Card (United States of America).

Non-government issued health-related cards such as private health insurance cards are also contemplated.

Non-health related government issued identification cards which are carried by the majority of patients may be utilized in some embodiments of the present systems. Documents such as a drivers' license, a concession card, a pension card, a national identity card, a passport, a taxation card, a utility bill and the like may be incorporated into the present systems.

Non-health related non-government issued cards such as a credit card (including chip-based cards), or a banking card are also contemplated.

Information from a third party card may be read by the patient identification means by laser, optical recognition, or magnetic means as discussed supra.

It will be appreciated that biometric means may also be utilized in the present systems. Unique (or virtually unique) identifiers such as finger prints, facial features, voice recognition, iris pattern and the like are contemplated. Hardware and software for exploiting biometric identification means are well known, and within the ability of the ordinary skilled person to incorporate into the present systems.

The present systems further include an electronic database of the medical practice. The database comprises patient-specific information such as name, contact details, preferred practitioner (a specific practitioner, or preferred gender, language spoken), appointment details (past, present, and future), billing details, social security status, health insurance details, and the like.

In one embodiment the system is configured such that upon identification of the patient, the system interrogates the database to retrieve patient-specific information. Such information may be imparted to the patient for confirmation or alteration. For example, the patient may be requested (via the display means) to confirm that they are the person identified by the system. The date of birth retrieved from the database may be displayed to the patient of a touch screen, with the patient touching a “yes” button on a touch screen if the date is correct.

Other details may be confirmed at this step. For example, contact details or social security status may be presented and confirmed by the patient if correct. If incorrect, the patient may proceed to have the details amended by a practice staff member manually or may enter the corrections required by way of touch screen input. Patient-initiated corrections may directly alter the relevant database entry, or more typically reviewed by a staff member before any alteration to the database is made. It is vital for patient details to be accurate such the follow up letters, reminders and the like are sent to the correct address. Accordingly, in a typical medical practice significant resources are spent on ensuring the accuracy of patient-specific information. Even with the best efforts of practice staff, patients will neglect to provide updated details unless specifically prompted to. The present systems may be configured to automatically request confirmation of details at every patient visit.

Retrieval of patient-specific information may be of further utility in the patient arrival process. In one embodiment the patient-specific information includes the last medical practitioner consulted by the patient, or the practitioner most commonly consulted by the patient. In larger, multi-practitioner practices lack of continuity of care can adversely affect patient experience or outcome. Where a patient is seen by different practitioners, the patient may need to provide the same information multiple times. Furthermore, each practitioner may have a less than complete picture of the patient's health given a complete reliance on the documented history only. Details of a historical nature such as the practitioner consulted are generally obtainable only by reference to patient histories. Non-medical and non-nursing staff are typically not permitted access to a history (and in some jurisdictions are legally prohibited access) and so in the absence of the present system a reception clerk will be unable to identify the practitioner who has previously cared for the patient. In many cases, the patient is unable to name a practitioner previously consulted sometimes due to a lack of familiarity with foreign names, or mental impairment such as memory problems.

Retrieval of database information at patient arrival can also retrieve appointment information (where the patient has an appointment). This may be useful where the patient has arrived at the wrong time or on the wrong day. The visual display means may inform the patient of the correct appointment details.

Where the database appointment information shows that the patient has an appointment, the visual display means may inform the patient of useful information such as the expected waiting time, the room or area in which the patient should be seated, or the name of the practitioner who will be consulting. Where a preference for a practitioner has not been previously specified, the visual display means may request patient input such as gender, language spoken, availability etc.

Where the database appointment information shows that the patient does not have an appointment then the visual display means may request a preference for a practitioner (as discussed supra). Alternatively, the patient may simply elect to request the next available practitioner in an effort to limit waiting time. Boolean preferences may be entered, such that the patient could select the next available male practitioner, or the next available female practitioner fluent in Cantonese. Customization of an appointment in this manner may be beyond the scope of some reception staff. Language issues and computer familiarity are two factors that may inhibit or protract more complex appointment customization.

The system may be further configured to inform the client as to the waiting time for a preferred practitioner.

As will be appreciated, systems capable of providing complex appointment options as discussed above may be in operable connection with a further system tracking the progress of practitioner's through their appointment list for the session. Typically, a client computer in the practitioner's consulting room is configured to allow the practitioner to transmit a signal to the main system server that a consultation has ended.

It will be further appreciated that the further system must record information on each practitioner such as gender and language ability to allow searches to be conducted based on patient preference.

Applicant further proposes that client arrival provides an opportunity to provide or request further information of the patient. The questions presented and/or information may be general in nature or may be patient-specific. In the latter case, patient-specific information may be retrieved from the database and utilised by algorithmic means to identify relevant questions and/or information.

In terms of exemplary general information, the visual display means may alert the patient to the upcoming influenza season. A general question may be presented as to whether or not the patient wishes to receive an influenza vaccination during their consultation. The patient response (“yes” or “no”) is forwarded electronically by the system to the practitioner for display on his/her client computer so as to remind the practitioner to offer vaccination during the consultation. In this way, a patient health outcome is improved (i.e. a protective vaccination at the correct time of year). Furthermore, an improved business outcome is provided for the medical practice. Busy practitioners may forget to offer a further service or product which is appropriate to the patient in the course of a consultation.

In terms of exemplary specific information, the patient-specific information retrieved from the database may include a positive asthmatic status. The patient may be presented with a patient-specific request to indicate a willingness to be involved in a clinical trial of an adjunct therapy to their normal asthma medication. If the patient agrees, the practitioner is electronically prompted by his/her client computer to discuss the trial during the consultation.

The present system is configured, in some embodiments, such that the output means presents general or patient-specific information, or presents general or patient specific questions of a financial nature at arrival.

In terms of exemplary general financial information, the visual display means may alert the patient of a fee payable for the upcoming consultation. The fee may be varied according to the time of day (with higher fees payable at higher demand times of the day, after hours, or on a weekend), a speciality of the practitioner (a specialist in cosmetic procedures may charge a higher rate than a general practitioner), or a standard gap fee payable by all non-exempt patients. The system may request the patient to confirm (by way of touch screen activation) acceptance of the fee. Where the fee is not accepted, the display means may inform the client that the appointment has been cancelled.

In terms of patient-specific financial information, the visual display means may inform that the patient has an outstanding account with the practice and that they must proceed to reception to organize payment. Where payment is not forthcoming, the appointment may be cancelled. The status of the patient's account is found by reference of the system to patient-specific financial data on the patient database.

In some embodiments, the system is configured so as to accept payment from a patient. An arrival kiosk may comprise internet based means for payment such as by PayPal™, EFTPOS, credit card, debit card, or cash. Alternatively, the kiosk may comprise card reading means (such as a magnetic reader or chip reader), or other means (such as a touch screen) by which a payment can be made

In an exemplary use of the system, after registration at the kiosk the patient is requested by way of the kiosk visual display to make an electronic prepayment (for example by swiping or “touching” a credit card to a dedicated device proximal to the kiosk), or a cash payment (by way of a bank note or coin deposit machine). This payment may be a base payment, a proportion of the expected fee, or a minimum fee, or a maximum fee, or a surcharge fee, or a facility fee. This amount may change from practitioner to practitioner, or from day to day, or be dependent on the time of day or the age of patient, or a concession status for example.

Where the prepayment is a surcharge fee the patient is informed of that by the visual display of the kiosk and also the reason (such as “weekend consultation surcharge” or “gap fee charge”). The fee may be displayed on the kiosk visual display, along with the reason, and optionally an explanation of the fee structure of the medical practice. In one embodiment, the pre-payment is not directed for any medical service, and may be a facility fee or other surcharge.

The system may be configured to provide a patient with the option to proceed (or to not proceed) with securing the consultation proceeding given the need for a prepayment.

Where charged, the amount charged as prepayment is stored in the system.

At the conclusion of the consultation, the practitioner charges the amount for the consultation and the pre-payed amount is added to the patient account and a receipt is printed out at the practitioner's desk for the full amount of the consultation in the name of the patient.

As an option to the foregoing, the system is configured to allow the practitioner to reverse the pre-payment, and print a receipt having only the amount deemed chargeable by the practitioner. For example, the prepayment may have been a gap fee (to cover the short fall between the payment provided by a government or insurer, and the actual consultation fee) and the practitioner may decide for any reason (economic, patient relationship or otherwise) to not levy a gap fee and charge only the fee that is payable by the government or insurer.

Where the prepayment is not reversed, the full amount of the consultation (with or without refund of prepayment) is payable. In some circumstances, a government or insurer may settle at least part the invoice such as the part that is covered by the government or insurer. The settlement may be made directly to the practice, or indirectly via the patient.

In any event the system may be configured such that the full amount of the consultation (i.e. any pre-payment as well as a charge levied by the practitioner at the end of the consultation) is shown on the receipt.

Systems configured to accept pre-payments, and optionally configured to allow a practitioner to refund in part or full a pre-payment provide advantage in so far as the need for a patient to attend reception to make a prepayment, or to seek a refund of a prepayment is lessened or abolished. Furthermore, the present systems may render unnecessary the need for a medical practice to maintain cash on site.

### Follow-Up appointments

The present systems may comprise means for allowing a patient to set a follow-up appointment after a consultation. It is often the case that a practitioner will wish to review a patient's progress or a pathology result at some future time. In some embodiments, no further hardware components are added to the system, with the patient utilising the same kiosk as used on arrival. More typically, the system comprises a dedicated follow-up appointment kiosk for patient use after a consultation. Generally, this follow-up kiosk will be located in the waiting area of the clinic. It will be appreciated that a second kiosk is not essential, and that the first kiosk may assume the function of setting a follow-up appointment.

The follow-up appointment kiosk may be configured to utilize patient identification means of the types disclosed supra for the arrival kiosk. In some embodiments, the follow-up appointment kiosk is configured so as to allow for entry of practitioner preferences by the patient, including complex Boolean filtering of preferences as disclosed supra for the arrival kiosk.

However, this second kiosk may provide for even greater complexity allowing for the patient to consider a range of other appointment parameters in the course of selecting a follow-up appointment. For example, alternative days and times of the appointment can be reviewed for one or more practitioners. The patient may search for an appointment with the practitioner just consulted on a week day afternoon at a time when no gap fee is payable. The display means may display a number of appointment options, with the client selecting a convenient date and time.

Alternatively, the patient may browse a practitioner's entire schedule in calendar form and select a convenient date and time. Complex Boolean search may also be incorporated into the follow-up kiosk, for example to allow a patient to avoid appointment times where are surcharge is payable, or to identify times when a female doctor is in attendance.

In making a follow-up appointment, the system may be configured such that the patient need not remember the practitioner's name, this information already being stored in the patient database and extracted when the patient schedules a follow-up appointment on the same day.

In some embodiments, the system is configured to read information from a document provided to the patient during the consultation. For example, the practitioner may hand the patient a document related to an ancillary medical or paramedical service such as a referral to a specialist practitioner or a pathology service. The document reading means may be any referred to elsewhere herein including bar code and QR code.

In one embodiment, the practitioner prints a medical or paramedical test request form during the consultation. For example, test request form may be a pathology request form requesting a full blood analysis, or a form requesting electrocardiograph testing, or a form requesting hire of a 24 hour blood pressure monitoring system. The document provided to the patient during the consultation may be a referral letter (for example a letter to a medical specialist, a speech pathologist, a physiotherapist, a counsellor, a social worker and the like). Where the letter is sealed in an envelope, the envelope may have the bar code or QR code affixed or printed thereonto.

In some embodiments, the document provided to the patient during the consultation may be dedicated only to facilitate the making of a follow-up appointment. The document may specify in computer-readable text, code or other means a time period after which a follow-up appointment may be made, or may instruct the system to retrieve the time period from a computer memory.

In some embodiments, the document provided to the patient during the consultation, may be an accounting document such as an invoice or a statement of account.

In some embodiments, the document provided to the patient during the consultation may be an electronic document (such as a text file) transmitted electronically to a patient device, such as a smart phone.

The form may be imprinted with a QR code containing patient-identifying information capable of being read by the present system, and/or information identifying the practitioner who issued the form, and the date by which the test should be completed and the date the results should be returned to the practitioner.

Alternatively, the system may be configured so as to recall information from a database pertaining to the requested test. For example, the QR code may identify the requested test as a full blood analysis with the expected turn-around time for that test being stored in a database and recalled by the system so as to provide a date by which the results could be expected to be returned to the practitioner.

In some embodiments, upon exit from the consultation the patient proceeds to a pathology collection room where a sample is collected. Once collection is completed, the patient proceeds to the follow-up kiosk where the QR or bar code is scanned, and open appointments on days after return of the pathology results are presented to the patient for selection.

In another embodiment, the practitioner prints a document encoding the practitioner's name and a date range for a follow up appointment. This embodiment may be useful where a follow-up appointment is to be delayed for some time, but within a maximum time limit. As an example, a patient may be prescribed new medication for hypertension with the practitioner wishing to review the patient's response within the month, however requires the patient to be taking the medication for at least a week in order properly assess the response. In this circumstance, the system will display to the patient all available appointments in weeks 2, 3 and 4 after the present consultation.

It will be appreciated that patients can be confused by a practitioner's instructions regarding when to book a follow-up appointment. In prior art methods, the patient returns to reception and requests a further appointment. The patient's request may not be in accordance with the practitioner's instructions and an appointment that is too early or too late is booked.

In some embodiments, the follow-up appointment means may not be a kiosk within the clinic. For example, the patient may book a follow up appointment from a home or workplace personal computer via an Internet browser, or utilize application software on a smart phone.

In any event, the follow-up appointment means may be configured to print an appointment confirmation document (such as a card or slip) or later reference by the patient. In some embodiments, the system is configured to transmit an electronic meeting request to the patient's email address (as retrieved from the database) allowing importation of the appointment into the patient's electronic calendar (such as Microsoft Outlook™). Alternatively, a local message via Bluetooth™ may be transmitted directly from the kiosk to the patient's smart phone. A medical practice-specific application software stored in the smart phone may receive the message and issue an alarm to remind the patient of the impending appointment. In another alternative, the system may dispatch an SMS text message to the patient's smart phone as a reminder.

### Triage Function

In one embodiment, the system is configured so as to triage patients. This embodiment is more so applicable to patients that have arrived without an appointment, and is typically executed at an arrival kiosk. A series of multiple choice questions may be proposed via the display means, the questions being decided by algorithmic means. Preferably, the questions are easily answerable by a lay person. For example, simple questions such as : “are you in severe pain?”, “have you suffered trauma?”, “are you having trouble breathing?”, “are you over 70 years of age?”, or “is the patient an infant?” are easily answered and will provide (again via algorithmic means) an index of the urgency by which a patient should be seen.

The system is configured in such embodiments to ensure patients in greatest need are seen in preference to patients with less urgent medical problems.

Given the benefit of the present specification, the skilled person (being entirely familiar with computer system architecture) is able to practice any aspect of the present invention.

For security and patient confidentiality the system is configured as private network, albeit with the ability to transmit and receive data to external networks (such as the Internet). As used herein, the term “private network” is intended to mean a computer network that is configured to decline access to individuals outside the medical practice. In one embodiment, the network is not accessible to the internet except by way of password, biometric contrivance, or other security mechanism. In one embodiment of the system the private network of the organization is configured such that it is not accessible by a processor-enabled device that is not owned, operated, controlled or administered by the medical practice. In one embodiment, the private network of the medical practice is implemented substantially completely within a firewall of the organization.

The network of the present systems may be implemented by wired means, such as twisted pair wire, computer networking cabling (wired Ethernet as defined by IEEE 802.3), coaxial cable, ITU-T G.hn technology, optical fiber. The term “wired” and its derivatives may be used to describe circuits, devices, systems, methods, techniques, communications channels, etc., that may communicate data through the use of modulated electromagnetic radiation through a solid medium.

In addition or alternatively the network may be implemented by wireless means including terrestrial microwave, communications satellite, cellular and PCS systems, radio and spread spectrum technologies such the IEE 802.11 protocols. In the context of this document, the term “wireless” and its derivatives may be used to describe circuits, devices, systems, methods, techniques, communications channels, etc., that may communicate data through the use of modulated electromagnetic radiation through a non-solid medium.

The network may implement any suitable communications protocol including an ethernet protocol, TCP/IP (including IPv4 and IPv6), Synchronous Optical Networking (SONET) and Synchronous Digital Hierarchy (SDH).

The network may be implemented at any desired scale including personal area network, local area network, storage area network, campus area network, backbone network, metropolitan area network, wide area network, enterprise private network, virtual private network, virtual network, or internetwork.

As mentioned supra, a preferred embodiment of the system is implemented partially or completely within a firewall of the organization. The firewall may be either software-based or hardware-based (or a combination). The firewall may operate by network layer or packet filter, application layer, proxy, or network address translation.

One embodiment of the systems and methods described herein is in the form of a computer-readable carrier medium carrying a set of instructions, e.g., a computer program for execution on one or more processors. Thus, as will be appreciated by those skilled in the art, embodiments of the present invention may be embodied as a method, an apparatus such as a special purpose apparatus, an apparatus such as a data processing system, or a computer-readable carrier medium. The computer-readable carrier medium carries computer readable code including a set of instructions that when executed on one or more processors cause a processor or processors to implement a method as described herein. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

Any information transmitted or received over a network of the system may be via a network interface device. While the carrier medium is shown in an example embodiment to be a single medium, the term “carrier medium” should be taken to include a single medium or multiple media (e.g., a centralized or distributed database, and/or associated caches and servers). The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media.

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

Implementation of the present invention may require the use of software program steps not hitherto implemented. For example, the software may instruct hardware to facilitate reversal of a prepayment for a medical service or a non-medical service at the instruction of a practitioner operating a medical centre billing system. Such reversal require programing steps of calculating a new account amount (debit or credit) and optionally instructing an electronic credit to a patient bank account or credit card account, or the reversal of a debit to patient bank account or credit card amount.

As another example of novel software program steps is the instruction of generating a text, code or other means in relation to a document issued by the practitioner of a medical centre appointment system to facilitate a follow-up appointment. Such instruction may direct the printing of a code or text onto a referral letter, pathology request form, account or other document issued in the course of a medical consultation.

The database of the present systems may be of any type capable of performing the functions described herein. Generally, the database is of the relational type, such as Advantage Database Server, Altibase, Apache Derby, Cubrid, Datacom, DB2, Drizzle, Empress Embedded Database, FileMaker, Firebird, HSQLDB, H2, Informix Dynamic Server, Ingres, InterBase, LucidDB, MAriaDB, MaxDB, Microsoft Access, FoxPro, Microsoft SQL Server, MonetDB/SQL, mSQL, Nexusdb, Omnis Studio, OpenLink Virtuoso, Oracle, Oracle, Rdb, Paradox, Peoplesoft, Pervasive PSQL, Polyhedra DBMS, Postgre SQL, RDM embedded, RDM server, SAP, ScimoreDB, smalISQL, SQL Anywhere, SQL Base, SQLite, Unidata and Xeround Cloud Database.

In a preferred form of the system, the database is based on data or a physical database that is ordinarily in place in a medical practice. Medical practices typically obtain and retain patient data such as name, address, date of birth, email address, billing information and the like. Some practice also incorporate computer-based patient histories, accounting and appointment management software. Thus, in some embodiments the present systems are incorporated so as to integrate with, overlay or otherwise form operable communications with prior-art computer systems and software of a medical practice. Exemplary prior art medical practice software and systems include: prognoClS, NueMD, Office Practicum, Kareo, WRS Health, MediTouch PM, Chirotouch, Electronic Medical Assistant, Velocidoc, WebPT, ADP AdvanceMD, ECLIPSE, PT Practice Pro, Nextech, Centricity, CareCloud Central, qualifacts, praxis EMR, Greenway, eClinicalWorks, ACOM Health RAPID, athenaClinicals, iO Practiceware, IntelleChart, The Digital Office, iSALUS EHR, MDCOnnection, TotalMD, PayDC, A.I.med, ChartLogic EHR Suite, CureMD, Therabill and iDoc, coreplus, boxtech, Shexie, Stat Health, Genie, Pracsoft, Medical Director, Jayex products such Enlighten, and Practix.

The systems may also be configured to integrate with, overlay or otherwise communicate with systems and software not specific to medical practices such as accounting software packages including MYOB, Xero, QuickBooks, FreshBooks, SageOne, Yendo, FeeAgent, AccountEdge, Less Accounting, Zoho and the like.

In another aspect, the present invention provides a method for managing a patient of a medical practice, the method comprising the steps of: providing the system as described herein, and instructing or allowing a patient to utilise the patient identification means.

The step of providing the system may necessitate consideration of the configuration and physical positioning of hardware component(s) of the system. The more important may be the patient identification means and/or the output means and/or the input means. Generally, the means are configured as a unitary contrivance, such as a kiosk, positioned such that upon entry a patient is very likely to see or hear the output means. In a preferred embodiment, the kiosk comprises a touch-sensitive video display screen (for input and output), and an optical reader and/or a magnetic reader.

The step of instructing the patient may be by way of the output means (such as a video display, or an audio output), or by means completely removed from the system (such as signage means).

Some of the embodiments are described herein as a method or combination of elements of a method that can be implemented by a processor of a processor device, computer system, or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

It will be understood that the systems or methods discussed are performed by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

It will be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following are hereby expressly incorporated into this Summary section, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

The present invention will now be more fully described by reference to the following non-limiting preferred embodiments.

### PREFERRED EMBODIMENTS OF THE INVENTION

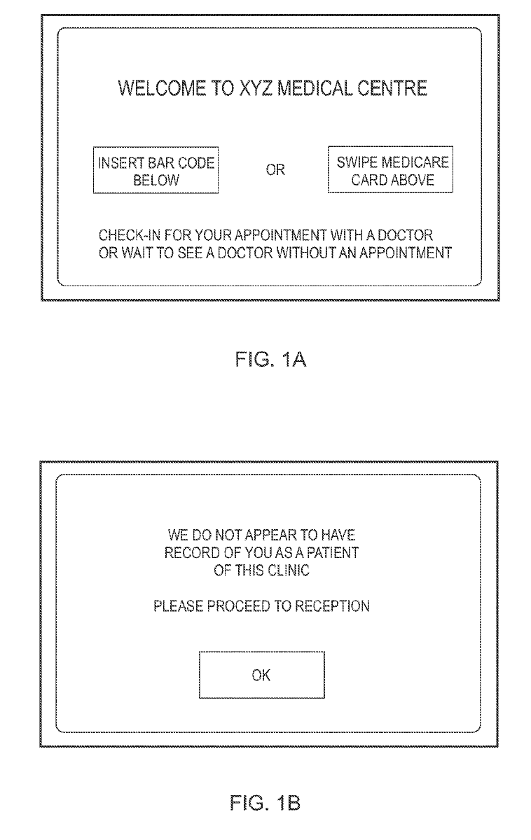

A patient presents at an arrival kiosk, whereby the display screen instructs the patient to either swipe a government issued health card or other document carrying a patient identification QR code (FIG. 1A).

If the patient has not previously attended the medical practice he/she is instructed to proceed to reception (FIG. 1B).

### Where patient has appointment

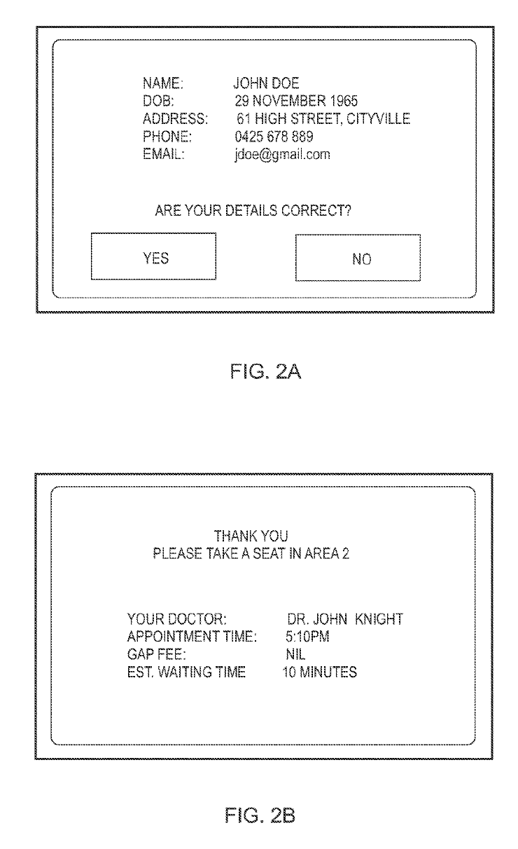

If the patient has an existing appointment, his/her details are displayed on the video display (FIG. 2A). If the details are correct he/she touches the “yes” button on the display screen (which has touch capabilities). If incorrect, the patient is directed to proceed to the reception desk where any errors are manually corrected by the reception clerk. Upon correction, the patient returns to the arrival kiosk.

Where the “yes” button of FIG. 2A is touched, a final arrival screen (FIG. 2B) is displayed confirming: arrival recorded, which room the doctor is consulting, how late the doctor is running, and whether a fee need be paid for the consultation. Further details of the final screen are discussed infra.

If more than one patient on the swiped card has an appointment, a screen with all the appointments for patients on that card is shown. The patient is prompted to click on their name to proceed.

If the patient has already checked in via the kiosk, tries again a message is shown that they have already arrived and to please take a seat.

Where the patient is more than 60 minutes early for their appointment, the display prompts the patient to attend reception. The patient may be able to secure an earlier appointment due to cancellation of another patient.

Where the patient is more than 15 minutes late for their appointment (or 10 minutes late within the last 3 appointment of the consulting practitioner's shift) the display prompts the patent to attend reception. It may be necessary for the patient to see another practitioner, or to book a replacement appointment.

If the patient arrives and the system (or a second system) records the doctor as “Sick or Away”, the display prompts the patent to attend reception. It may be necessary for the patient to see another practitioner, or to book a replacement appointment.

### Where the patient has no appointment

If the patient does not have an existing appointment (often termed a “walk-in” patient), this is automatically recognised upon successful identification of the patient given that the database entry for that patient will not have stored an appointment

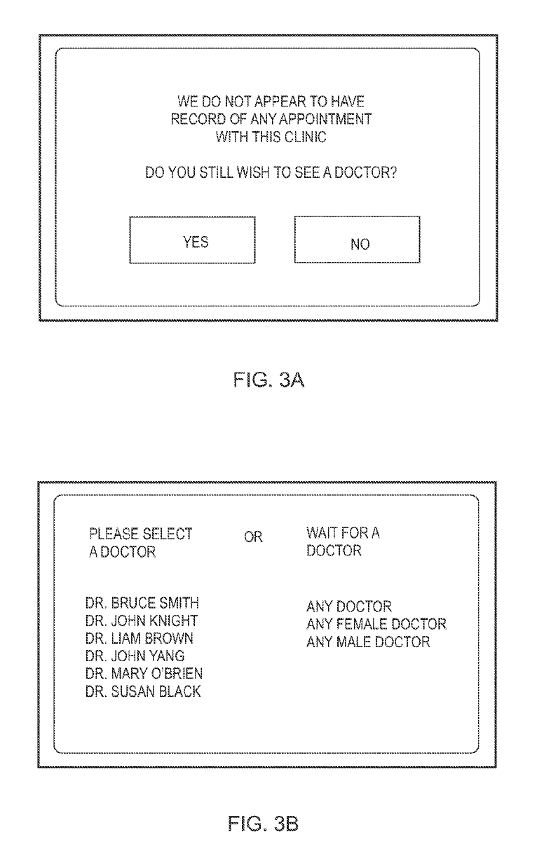

The display informs the patient that he/she has no appointment and requests if he/she still wishes to see a doctor (FIG. 3A).

Where the patient touches “yes” screen 3B is displayed to select either: a specific doctor, or the next available doctor, or the next available male/female doctor.

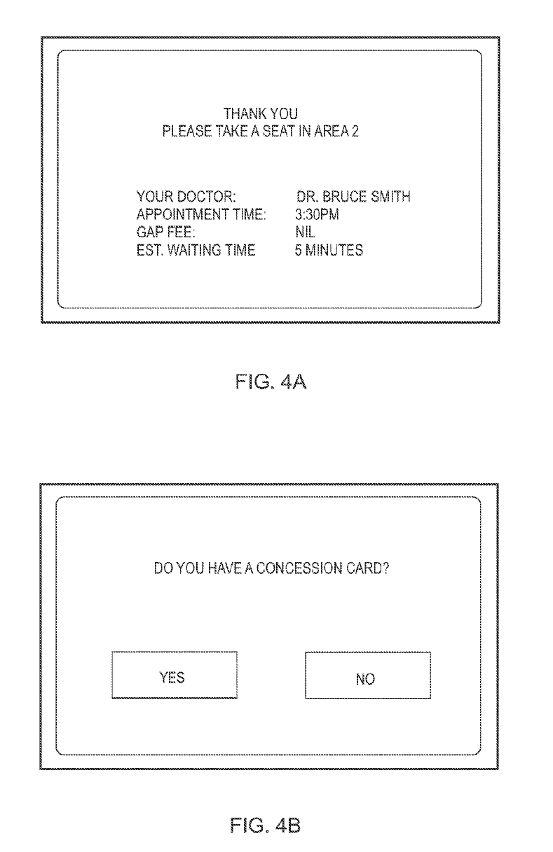

The display then requests the patient to move to the waiting area. Confirmation of the consulting doctor, location of consulting room, the expected waiting period and any fee payable is also displayed (FIG. 4A).

The system is configured such that a given patient can only make one walk-in request to see a doctor per day.

The system is further configured such that a walk-in patient is not able to secure an appointment for a practitioner outside the practitioner's shift hours, during breaks (such as a lunch break), within the last hour of practice opening hours, within the last hour of a practitioner's shift, or for a practitioner who declines walk-in patients.

The system is configured such that these preferences (which are set in favour of the practitioner and/or medical practice) may be altered at any time within the system, or by alteration of a parameter utilised by a second system (such as a practitioner rostering system).

### Query for Patients Regarding Concession Status

The patient is requested whether they are in possession of a concession card (FIG. 4B). In some health care regimes, this is an important consideration given the higher rebate where a patient is able to receive a concession.

Depending on the regime under which the medical centre operates, the issue of concession status may only be relevant where a gap fee is payable. As discussed elsewhere herein, a gap fee may be payable at peak times. Accordingly, questions regarding concession status by the system may only be presented for an appointment where a gap fee is payable.

Where the patient indicates a positive concession status, the system is configured to interrogate the patient database to identify whether or not the relevant card (such as a health care card, or a pension card) is registered at the clinic. Where the card is not registered, the display prompts the patient to attend reception so as to complete the registration process.

### Arrival Final Screen

Irrespective of the above considerations, the patient (where they have an existing appointment, or have entered as a walk-in patient and arranged an appointment), an arrival final screen is displayed.

A gap fee may be displayed in accordance with the practice fee structure according to day, time, etc.

The display shows the expected waiting time is displayed for patients with appointments: waiting time “up to . . . .”

The display shows the number of minutes early for appointment plus set value (if minutes early >15)

The waiting time is calculated as follows:
0 patients arrived with appointment for the consulting practitioner: up to 10 mins+minutes early
1 patient arrived: up to 15 mins+minutes early
2 or 3 patients arrived: up 25 mins+minutes early
>3 patients arrived: up to 40 mins+minutes early

For walk-in patients the expected waiting is calculated as follows:
0 or 1 walk-in patients waiting: up to 30 mins
2 or 3 walk-in patients waiting: up to 40 mins
>3 walk-in patients waiting: up to 60 mins

A flashing room is displayed on a practice map if the patient has an appointment with a set practitioner. The system is configured so as to interrogate a second system (such as a practice management system) to identify the relevant room.

### Debtor assigned to patients paying a gap fee.

Where the patient is >16 years old and has paid a private fee before at the practice, the system is configured to mark the patient as the debtor.

Where the patient is >16 years old and has not paid a private fee before at the clinic, the system is configured to mark the patient as a debtor and creates a new debtor in the practice billing system.

Where the patient is <16 years old and has paid a private fee before at the practice and a parent/guardian has been assigned as the debtor, the system is configured to mark the parent/guardian as the debtor.

Where the patient is <16 years old and has not paid a private fee before at the practice, the patient is created as the debtor in the billing system and is marked as the debtor. The parent/guardian is then assigned as the debtor when they return to reception to pay for the consultation

### Where patient requires follow-up appointment

Where the patient must visit pathology after the consultation, a bar code is printed at the foot of the request form handed to the patient. The patient proceeds to pathology collection, and then moves to the follow-up appointment kiosk.

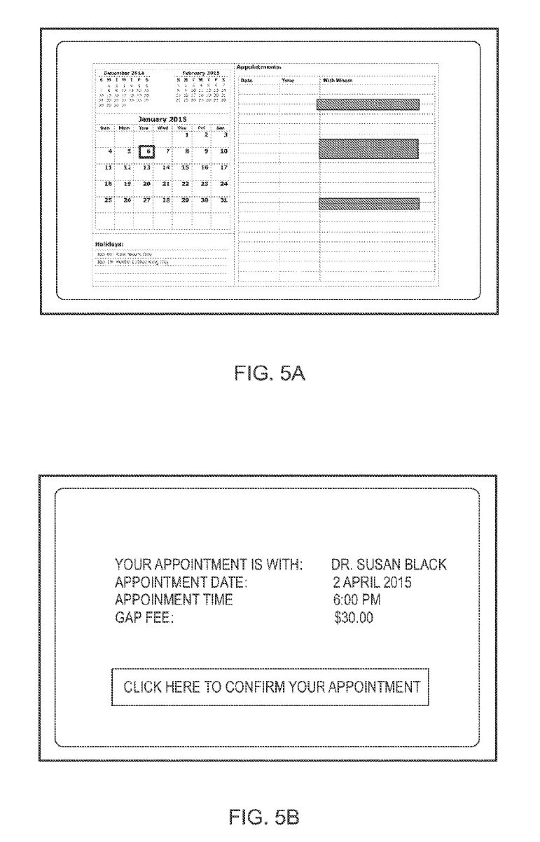

At the follow-up kiosk, the pathology slip barcode is read and displays the doctor's appointment schedule to allow the patient to select by touch a convenient date and time. The appointment is made in real time (FIG. 5A).

The display confirms the appointment, allowing the patient to confirm (FIG. 5B).

The patient confirms the appointment and an appointment card is issued.

A group of 80 participants (selected so as to be representative of the demographics of patients attending a busy multi-practitioner medical practice) trialled the system described supra. A median arrival processing time of 15 seconds was noted. The number of patients requiring assistance of reception staff was reduced by 55%.

Moreover, the majority of participants reported a preference for using the arrival kiosk over interaction with reception staff. The speed of arrival using the kiosk was cited as important, as was the lowered possibility of reception staff discussing matters of a private nature with the participants.

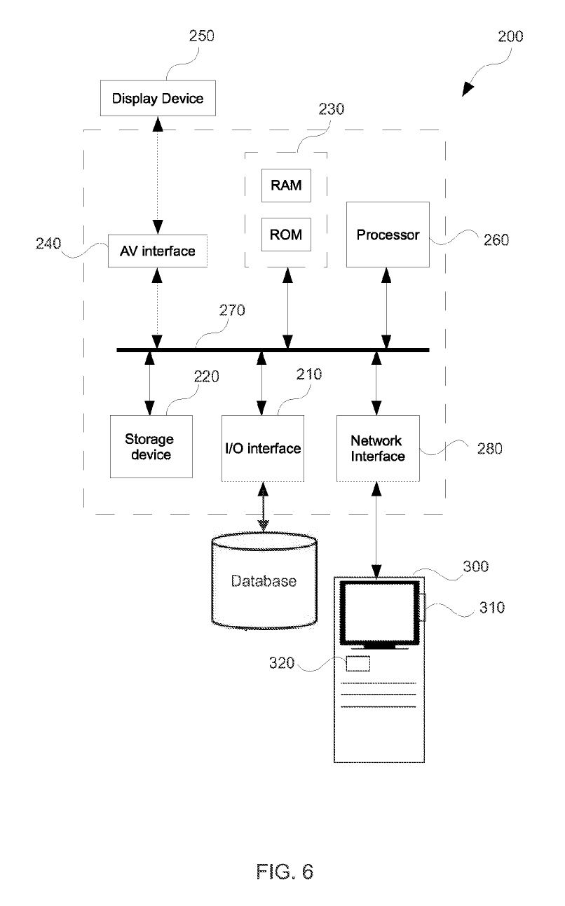

Reference is now made to FIG. 6 which is a block diagram showing a preferred embodiment of the system comprising a central database 100 having a plurality of patient records, each record having information on a patient. The database is operably connected to a central computer 200 via an I/O interface 210, the computer having a local storage device 220 and memory 230 having computer-executable instructions for executing the computer-executed actions required, an AV interface 240 operably connected to a display device 250, and a processor 260, all operably connected to a communications bus 270. The system includes a kiosk 300 having a magnetic reader 310 and QR code reader 320, the kiosk 300 being operably connected to the central computer 200 via a network interface 280. Both central computer 200 and kiosk 300 are disposed within a firewall of the organization.

1. A system for managing a patient of a medical practice, the system comprising:
patient identification means,
a database comprising patient-specific information,
a processor-based device in operable connection with the patient identification means and the patient database, and
patient-viewable or patient-audible output means.

2. The system of claim 1 wherein the patient identification means is configured to allow identification of the patient without the need for patient input.

3. The system of claim 1 wherein the patient identification means is capable of reading information from medical practice-issued identification means or third party-issued identification means.

4. The system of claim 1 wherein the patient identification means is capable of reading information from a personal electronic device of the patient.

5. The system of claim 1 wherein the information obtained from the patient identification means is transmitted to the processor-based based device, the processor based-device interrogating the patient database to retrieve patient-specific information.

6. The system of claim 1 wherein the patient-viewable display means displays general and/or patient-specific information.

7. The system of claim 5 wherein the patient-specific information and/or general information relates to an appointment time, an appointment location, a practitioner identity, a practitioner characteristic, a patient preference, a fee, a suggested product or service, or a health alert.

8. The system of claim 5 wherein the patient-specific information relates to a patient name, a patient contact detail, or a patient health insurance detail.

9. The system of claim 1 comprising patient-usable data input means, wherein the patient-viewable display means is configured to display a question and the patient-usable data input means is configured to receive a response by the patient.

10. The system of claim 9 wherein the question is selected from a group of existing questions based on the patient identity and/or patient-specific information of the database.

11. The system of claim 1 wherein the patient-usable input means and patient-viewable output means are configured to allow the patient to set a follow up appointment during or after a consultation.

12. The system of claim 1 further comprising means for reading information from a document related to an ancillary medical or paramedical service.

13. The system of claim 1 wherein the patient identification means is a configured as a kiosk located in a reception or entry of a medical practice, the kiosk comprising visual display means.

14. The system of claim 1 comprising payment means.

15. The system of claim 14 wherein the payment means is on, or proximal to the kiosk.

16. The system of claim 14 configured so as to receive a prepayment via the payment means.

17. The system of claim 16 configured so as to (i) store the prepayment electronically, (ii) allow a practitioner to enter a consultation charge, and (iii) calculating a total charge based on the amount of the stored prepayment and the consultation charge.

18. (canceled)

19. The system of claim 1 comprising (i) printing means configured to issue an appointment document to the patient, and/or (ii) data transmission means configured to electronically transmit appointment information to a patient computer or a patient personal electronic device.

20. A method for managing a patient of a medical practice, the method comprising:
providing a system of for managing a patient of a medical practice, the system comprising:
patient identification means,
a database comprising patient-specific information,
a processor-based device in operable connection with the patient identification means and the patient database, and
patient-viewable or patient-audible output means, and
instructing or allowing a patient to utilise the patient identification means.

21. (canceled)

22. An electronic kiosk comprising patient identification means, the kiosk operable within the system of claim 1.

23. (canceled)

24. (canceled)
